SEQUENCE LISTING (9 sequences)

SEQ ID NO: 1 - protein, 551 aa, Streptococcus pneumoniae
GSGAKRGTIYDRNGVPIAEDATSGGPNRSYPNGQFASSFIGGGMESSLNSILAGGGGDGKDVYTTISSPLQSFMETQMDAFQEKVKGKYMTATLVSAKTGEILATTQRPTFDADTKEGITEDFVWRDILYQSNYEPGSTMKVMMLAAAIDNNTFPGGEVFNSSELKIADATIRDWDVNEGLTGGRTMTFSQGFAHSSNVGMTLLEQKMGDATWLDYLNRFKFGVPTRFGLTDEYAGQLPADNIVNIAQSSFGQGISVTQTQMIRAFTAIANDGVMLEPKFISAIYDPNDQTARKSQKEIVGNPVSKDAASLTRTNMVLVGTDPVYGTMYNHSTGKPTVTVPGQNVALKSGTAQIADEKNGGYLVGLTDYIFSAVSMSPAENPDFILYVTVQQPEHYSGIQLGEFANPILERASAMKDSLNLQTTAKALEQVSQQSPYPMPSVKDISPGDLAEELRRNLVQPIVVGTGTKIKNSSAEEGKNLAPNQQVLILSDKAEEVPDMYGWTKETAETLAKWLNIELEFQGSGSTVQKQDVRANTAIKDIKKITLTLGD

SEQ ID NO: 2 - DNA, 46 bp, Artificial sequence
  Description of the artificial sequenceprimer
gtcgacttagtctcctaaagttaatttaatttttttaatgtttttg

SEQ ID NO: 3 - DNA, 21 bp, Artificial sequence
  Description of the artificial sequenceprimer
ggatccgggacaggcactcgc

SEQ ID NO: 4 - DNA, 43 bp, Artificial sequence
  Description of the artificial sequenceprimer
cataaatagtcccacgtttggccccggatccacgcggaaccag

SEQ ID NO: 5 - DNA, 51 bp, Artificial sequence
  Description of the artificial sequenceprimer
gtttgggtaactacgattgggacctccagaggttgcatcctcagcaatcgg

SEQ ID NO: 6 - DNA, 48 bp, Artificial sequence
  Description of the artificial sequenceprimer
gttcaaggaactctccattccaccgccgataaaactagaagcaaattg

SEQ ID NO: 7 - DNA, 49 bp, Artificial sequence
  Description of the artificial sequenceprimer
tgtataaacatccttaccgtccccacctccccctgcaagaatactgttc

SEQ ID NO: 8 - DNA, 30 bp, Artificial sequence
  Description of the artificial sequenceprimer
ccgcatatggccaaacgtgggactatttat

SEQ ID NO: 9 - DNA, 32 bp, Artificial sequence
  Description of the artificial s,quenceprimer
ggctcgagttagtctcctaaagttaatgtaat

(19) United States
(12) Patent Application Publication
(10) Pub. No.: US 2006/0003330 A1
(43) Pub. Date: Jan. 5, 2006

(54) Streptococcus pneumoniae pbp2x mini-protein and uses thereof

(75) Inventors: Otto Dideberg, Grenoble (FR); Thierry Vernet, Grenoble (FR); Nicolas Mouz, Grenoble (FR)

Correspondence Address: LOWE HAUPTMAN GILMAN AND BERNER, LLP, 1700 DIAGONAL ROAD, SUITE 300 /310, ALEXANDRIA, VA, 22314, US

(73) Assignee: COMMISSARIAT A L'ENERGIE ATOMIQUE CENTRE NATIONAL DE LA RECHERCHE SCIENTIF, PARIS (FR)

(21) Appl. No.: 10/520,655

(22) PCT Filed: Jul. 11, 2003
(86) PCT No.: PCT/IB03/03397

§ 371 Date: Mar. 7, 2005

(30) Foreign Application Priority Data
Jul. 11, 2002 (FR) 02/08724

(51) Int. Cl. C12Q 1/68; G01N 33/554; G01N 33/569; C07H 21/04; C07K 14/315; C12N 15/74

(52) U.S. Cl. 435/6; 435/7.32; 435/69.3; 435/252.3; 435/471; 530/350; 530/388.4; 536/23.7

(57) ABSTRACT

Modified recombinant protein derived from Streptococcus pneumoniae PBP2x, referred to as mini-PBP2x, consisting of a concatenation of the fragments corresponding, respectively, to the amino acids located between positions 74 to 90, 186 to 199, 218 to 228 and 257-750, with reference to the sequence of the PBP2x protein of the strain R6 (SWISSPROT P14677 or GENBANK 18266817), each one of said fragments being preceded by a peptide fragment of 1 to 7 amino acids, and uses of said mini-PBP2x protein for selecting and identifying antibiotics which are active on β-lactam-resistant strains of S. pneumoniae.

The present invention relates to a modified recombinant protein derived from Streptococcus pneumoniae PBP2x, referred to as mini-PBP2x, and also to uses thereof for selecting and identifying antibiotics which are active on β-lactam-resistant strains of S. pneumoniae.

Resistance to antibiotics represents a major problem in anti-infection therapy. For several years, the appearance of an increasing number of bacterial strains very resistant to the compounds of the β-lactam family (penicillins and cephalosporins, etc.), which represent the antibiotics most commonly used throughout the world for more than 60 years, has been observed; currently, 21% of clinical isolates of Streptococcus pneumoniae, one of the major pathogens of the upper respiratory pathways, are very resistant to β-lactams (minimum inhibitory concentration (MIC)>2 μg/ml; Doern et al., Antimicrob. Agents Chemother, 2001, 45, 1721-1729).

The target for β-lactams is PBPs (Penicillin Binding Proteins), membrane proteins which catalyse essential steps of the synthesis of bacterial wall peptidoglycan. Each bacterial species has several PBPs, the molecular weight of which varies between 30 kDa and 100 kDa.

The high molecular weight PBPs comprise a short cytoplasmic domain, a single transmembrane domain and a large periplasmic domain, and are divided up into class A (having both transpeptidase activity and glycosyltransferase activity (bridging)) and into class B (having an N-terminal domain of unknown function and a domain where the transpeptidase activity is located), which are readily identifiable by virtue of units of amino acids which are conserved for each class of PBP.

The low molecular weight PBPs essentially possess carboxypeptidase activity. Biochemical studies indicate that only the high molecular weight PBPs are multifunctional enzymes essential to bacterial survival; on the other hand, the low molecular weight PBPs are not essential and regulate only the degree of bridging of the bacterial wall.

The method of action of β-lactams is based on structural analogy between the ring of β-lactams and the D-alanyl-D-alanine of the C-terminal end of the peptides of the peptidoglycan; β-lactams are pseudosubstrates for the transpeptidase, capable of acylating the serine residue of the active site of this transpeptidase, which is then deacylated very slowly, thus disturbing peptidoglycan synthesis.

The mechanisms of β-lactam resistance in Gram⁺ bacteria comprise essentially: production of β-lactamases which hydrolyse the ring of β-lactams before they reach their target (PBPs), alteration of membrane permeability, and modification of PBPs.

Streptococcus pneumoniae has developed β-lactam resistance by modifying its PBPs; the combination of point mutations in the PBPs genes and of events of homologous recombination of these genes with those of related Streptococcus strains (S. mitis, S. oralis) results in the production of PBPs which have a low affinity for β-lactams.

Among the S. pneumoniae PBPs, the PBP2x protein, which is essential for the survival of S. pneumoniae and constitutes the primary factor of resistance to β-lactams (Hakenbeck et al., J. Bacteriol., 1998, 180, 1831-1840), represents the major target for identifying novel antibiotics which are active on β-lactam-resistant strains of S. pneumoniae.

PBP2x is a 750 amino acid protein comprising a cytoplasmic region (positions 1 to 18), a transmembrane region (positions 19 to 48), a non penicillin-binding domain (or n-PB, positions 49-265), a penicillin-binding domain/transpeptidase domain (positions 266 to 615) and a C-terminal domain (616-750).

Structure-function analysis of PBP2x has made it possible to specify the molecular mechanisms of the resistance of PBP2x to β-lactams (Mouz et al., P.N.A.S., 1998, 95, 13403-13406; J. Biol. Chem., 1999, 274, 19175-19180).

The 3-dimensional structure of PBP2x has been determined from a PBP2x comprising a deletion of the cytoplasmic and transmembrane regions, referred to as PBP2x*; this structure has been determined with a resolution of, respectively, 3.5, 2.4 and 3.2 Å, for a β-lactam-sensitive strain of S. pneumoniae (strain R6: Pares et al., Nature Struct. Biol., 1996, 3, 284-289; Gordon et al., J. Mol. Biol., 2000, 299, 477-485) and for a resistant clinical isolate (Dessen et al., J. Biol. Chem., 2001, 276, 45106-45112; accession numbers in the database PROTEIN DATA BANK (http://www.rcsb.org/), respectively 1PMD, 1QME and 1K25).

The three-dimensional structure of a complex between a PBP2x* of a sensitive strain (strain R6) and a β-lactam (cefuroxime) has also been determined with a resolution of 2.8 Å (accession number 1QMF in the data-base PROTEIN DATA BANK and Gordon et al., J. Mol. Biol., 2000, 299, 477-485).

However, the data currently available have not made it possible to identify novel antibiotic molecules which are active on β-lactam-resistant strains of S. pneumoniae, in particular due to the difficulty in obtaining crystals having a good diffraction power (2.5 Å) for native PBP2x (wild-type, sensitive to β-lactams), the PBP2x-inhibitor complexes and the β-lactam-resistant variants of PBP2x.

Specifically, analysis of PBP2x crystals which diffract at 2.4 Å (1QME, Gordon et al., J. Mol. Biol., 2000, 299, 477-485) has shown that they contain a form of PBP2x which is partially proteolysed at the peptide bond between the residues of positions 182 and 183, obtained under non-reproducible experimental conditions, resulting from exposure of the PBP2x protein at 25° C. for several months, during the crystallization process.

The inventors have investigated, choosing, as a model, PBP2x of a penicillin-sensitive strain of S. pneumoniae (strain R6), the 3-dimensional structure of which is known, whether it is possible to obtain PBP2xs which are more crystallizable in a reproducible manner and which have a better diffraction power.

They have thus noted that additional deletions in the region corresponding to the n-PB domain (positions 50 to 265 of PBP2x) which do not modify the enzymatic properties of PBP2x (binding to β-lactams and transpeptidase activity) make it possible to readily obtain crystals which have a better quality of diffraction.

The approach envisaged can be applied to any PBP2x derived from a β-lactam-sensitive or -resistant strain of S. pneumoniae.

This mini-protein represents a tool which is perfectly suited to selecting and identifying novel antibiotics which are active on β-lactam-resistant strains of S. pneumoniae.

Consequently, a subject of the present invention is a protein derived from a Streptococcus pneumoniae PBP2x, characterized in that it consists of a concatenation of the fragments corresponding respectively to the amino acids located between positions 74 to 90, 186 to 199, 218 to 228 and 257-750, with reference to the sequence of the PBP2x protein of the strain R6 (SWISSPROT P14677 or GENBANK 18266817), each one of said fragments being preceded by a peptide fragment of 1 to 7 amino acids.

The protein according to the invention is hereinafter referred to as PBP2x mini-protein or mini-PBP2x; it therefore comprises the deletion of amino acids located respectively between positions 1 to 73, 91 to 185, 200 to 217 and 229 to 256, as defined above, and the insertion, in place of said deletions, of a peptide fragment of 1 to 7 amino acids.

According to an advantageous embodiment of said mini-PBP2x protein, said peptide fragment of 1 to 7 amino acids comprises amino acids of the sequence of said Streptococcus pneumoniae PBP2x protein corresponding to those located between positions −1 to −7, relative to the residues of positions 74, 186, 218 and 257, and/or between positions +1 to +7, relative to the residues of positions 90, 199 and 228, as defined above.

According to another advantageous embodiment of said mini-PBP2x protein, said peptide fragment of 1 to 7 amino acids comprises amino acids in which the volume of the side chain is small, such as alanine (A), serine (S), glycine (G) or threonine (T).

For the purpose of the present invention, said S. pneumoniae PBP2x protein is defined by the following characteristics:
it is encoded by the gene referred to as pbpX, corresponding to that located in the genome of the S. pneumoniae strain R6, between positions 2263 and 4515 of the locus having the NCBI accession number AE008411 or the GENBANK accession number 15457852;

it comprises the following amino acid units (one-letter code), specific to the class B PBPs:

| | |
|---|---|
| M1: | RGXhX (D/S) RSGXXXA |
| M2: | (R/K) XXPXG |
| M3: | (G/Y) hEXXXDXXL |
| M4: | hXX (S/T) hDXXXQ |
| M5: | T (G/S) EhhXXXXSPXh (D/N) |
| M6: | hEP (A/G) SXXK |
| M7: | hXXSXNh |
| M8: | K (T/S) G, |

in which the amino acids in bold are strictly conserved in the sequences of class B PBPs;/represents an alternative, for example D/S represents an aspartic acid or a serine; X represents any amino acid; h represents a hydrophobic amino acid and the other letters represent the amino acids most commonly encountered at this position; and
its sequence exhibits, over its entirety, at least 30% identity, preferably at least 50% identity, or at least 85% similarity, with the sequence of the strain R6 (SWISSPROT P14677).

The identity of a sequence relative to a reference sequence is assessed as a function of the percentage of amino acids residues which are identical, when the two sequences are aligned, so as to obtain the maximum correspondence between them.

A protein which has an amino acid sequence having at least X % identity with a reference sequence is defined, in the present invention, as a protein whose sequence can include up to 100-X alterations per 100 amino acids of the reference sequence, while at the same time conserving the functional properties of said reference protein. For the purpose of the present invention, the term “alteration” includes deletions, substitutions or insertions, which are consecutive or dispersed, of amino acids in the reference sequence.

The similarity of a sequence relative to a reference sequence is assessed as a function of the percentage of amino acid residues which are identical or which differ by conservative substitutions, when the two sequences are aligned so as to obtain the maximum correspondence between them. For the purpose of the present invention, the term “conservative substitution” is intended to mean the substitution of an amino acid with another which has similar chemical properties (size, charge or polarity), which generally does not modify the functional properties of the protein.

A protein which has an amino acid sequence having at least X % similarity with a reference sequence is defined, in the present invention, as a protein whose sequence can include up to 100-X nonconservative alterations per 100 amino acids of the reference sequence. For the purpose of the present invention, the term “non-conservative alterations” includes the deletions, nonconservative substitutions or insertions, which are consecutive or dispersed, of amino acids in the reference sequence.

The present invention encompasses the mini-PBP2xs derived from a PBP2x of any β-lactam-sensitive or -resistant strain of S. pneumoniae, in particular of β-lactam-resistant clinical isolates. By way of nonlimiting example, mention may be made of the β-lactam-resistant strain C 506, which is described in the article in the names of Laible et al. (Mol. Microbiol., 1989, 3, 1337-1348).

According to an advantageous embodiment of said mini-PBP2x protein, it is derived from a β-lactam-resistant strain of Streptococcus pneumoniae.

According to another advantageous embodiment of said mini-PBP2x protein, it consists of the concatenation of the fragments, as defined above, of PBP2x of the β-lactam-sensitive Streptococcus pneumoniae strain R6 (SWISSPROT P14677) and it has the sequence SEQ ID No. 1.

According to another advantageous embodiment of said mini-PBP2x protein, it comprises a substitution of at least one methionine residue with a selenomethionine residue.

According to another advantageous embodiment of said mini-PBP2x protein, it is associated with a ligand, in particular in the form of a mini-PBP2x/ligand complex.

In accordance with the invention, said ligand consists of an organic molecule, in particular a protein such as an antibody, or an inorganic molecule; said ligand is in particular a substrate, such as a pseudosubstrate, capable of binding to said mini-PBP2x protein via its active site and of inhibiting the activity of said mini-PBP2x.

According to yet another advantageous embodiment of said mini-PBP2x protein, it is in the form of a crystal.

In accordance with the invention, said crystal consists of a mini-PBP2x in free form or associated with a ligand as defined above.

In accordance with the invention, the conditions for crystallization of the mini-PBP2x are determined by the suspended drop technique. For example, crystals of mini-PBP2x are obtained under the following conditions: mini-PBP2x (12 mg/ml), 100 mM sodium Hepes, pH 7.5, 2% V/V PEG 400, 2M ammonium sulphate, at a temperature of 8° C.

The mini-PBP2xs consisting of fragments of PBP2x from β-lactam-resistant strains of S. pneumoniae are of use for screening and identifying novel antibiotics; the mini-PBP2xs consisting of fragments of PBP2x from sensitive strains of S. pneumoniae, in particular the mini-PBP2x of sequence SEQ ID No. 1, are of use as a control, in screening and identifying antibiotics.

A subject of the present invention is also a peptide, characterized in that it consists of a fragment of at least 7 amino acids of the mini-PBP2x protein, as defined above, which peptide includes at least one residue chosen from those located at positions 74, 90, 186, 199, 218, 228 and 257, as defined above; such a peptide is of particular use for producing antibodies which recognize specifically mini-PBP2x.

For instance, if said peptide has 7 amino acids and comprises at position 3 the residue corresponding to position 74 of the mini-PBP2x protein as defined hereabove, therefore, said peptide begins at position 74−2 and ends at position 74+4 in reference to said mini-PBP2x protein and it has the following sequence: Ala-Lys-Arg-Gly-Thr-Ile-Tyr.

All these peptides are specific of the instant mini-PBP2x protein because they correspond to positions at the junction, i.e. they always comprise a fragment of 1-7 aminoacids as defined hereabove.

A subject of the present invention is also antibodies, characterized in that they are directed against a peptide as defined above.

In accordance with the invention, said antibodies are either monoclonal antibodies or polyclonal antibodies.

These antibodies can be obtained by conventional methods, known in themselves, comprising in particular the immunization of an animal with a protein or a peptide in accordance with the invention, in order to make it produce antibodies directed against said protein or said peptide.

Such antibodies are in particular of use for immobilizing the mini-PBP2x on a solid support, or else for co-crystallizing it in the form of antibody-mini-PBP2x complexes.

The subject of the present invention is also an isolated nucleic acid molecule, characterized in that it is selected from the group consisting of the sequences encoding a mini-PBP2x as defined above and the sequences complementary to the preceding sequences, which are sense or antisense.

A subject of the invention is also primers intended to specifically amplify the sequences encoding a PBP2x, characterized in that they are selected from the group consisting of the pair of sequence SEQ ID Nos. 2-3.

A subject of the invention is also probes and primers, characterized in that they comprise a sequence of approximately 10 to 30 nucleotides corresponding to that located at the junction of the peptide fragments of 1 to 7 amino acids and the fragments of PBP2x; these probes and these primers make it possible to specifically detect/amplify said nucleic acid molecules encoding a mini-PBP2x.

According to an advantageous embodiment of said probes and said primers, they have a sequence selected from the group consisting of the sequences SEQ ID Nos. 4 to 9.

The nucleic acid molecules according to the invention are obtained by conventional methods, known in themselves, according to standard protocols such as those described in Current Protocols in Molecular Biology (Frederick M. AUSUBEL, 2000, Wiley and son Inc, Library of Congress, USA).

The sequences encoding PBP2x can be obtained by amplification of a nucleic acid sequence by PCR or RT-PCR, or else by screening genomic DNA libraries by hybridization with a homologous probe. For example, they are amplified by PCR using a suitable pair of primers, such as the pair of sequences SEQ ID Nos. 2-3.

The derived nucleic acid molecules, encoding a mini-PBP2x, are obtained by the conventional methods, making it possible to introduce mutations into a nucleic acid sequence, known in themselves, according to the abovementioned standard protocols. For example, the sequence encoding the mini-PBP2x can be obtained by site-directed mutagenesis according to the method of Kunkel et al. (P.N.A.S., 1985, 82, 488-492), using the primers SEQ ID Nos. 4 to 7, and then PCR amplification using the primers SEQ ID Nos. 8 and 9, as defined above.

A subject of the present invention is also a recombinant vector, characterized in that it comprises an insert selected from the group consisting of the nucleic acid molecules encoding a mini-PBP2x and their fragments as defined above.

Preferably, said recombinant vector is an expression vector in which said nucleic acid molecule or one of its fragments is placed under the control of suitable elements for regulating transcription and translation. In addition, said vector may comprise sequences (tags) fused in-frame with the 5′ and/or 3′ end of said insert, of use for immobilizing and/or detecting and/or purifying the protein expressed from said vector.

Preferably, said expression vector is a prokaryotic vector.

These vectors are constructed and introduced into host cells by the conventional methods of recombinant DNA and genetic engineering, which are known in themselves.

A subject of the present invention is also cells transformed with a recombinant vector as defined above.

According to an advantageous embodiment of the invention, said cells are prokaryotic cells.

The recombinant vectors and the transformed cells as defined above are of particular use for producing mini-PBP2x and the derived peptides, as defined above.

A subject of the present invention is also the use of a mini-PBP2x as defined above, for screening and identifying antibiotics.

According to an advantageous embodiment of the invention, said screening is carried out by a method comprising at least the following steps:
a₁) bringing a mini-PBP2x as defined above into contact with a test substance,
b₁) detecting, by any suitable means, the binding of said test molecule with the mini-PBP2x and/or the inhibition of the activity of said mini-PBP2x resulting from this binding, and
c₁) selecting the active substances capable of binding to the mini-PBP2x and/or of inhibiting the activity of said mini-PBP2x, which can be used as antibiotics.

The binding of said test molecule with the mini-PBP2x can be measured using conventional binding assays which make it possible to detect molecules capable of binding covalently at the active serine (S₃₃₇, with reference to the sequence of PBP2x of the strain R6), in particular using a ligand pre-labelled with a chromophore or with a fluorophore, such as a cephalosporin coupled to a chromophore (nitrocefin), or else by measuring the decrease in intrinsic fluorescence of said mini-PBP2x, as described in the article in the names of Jamin et al., Biochem. J., 1993, 292, 735-741.

The inhibition of the enzymatic activity of the mini-PBP2x can be determined either by measuring the inhibition of hydrolysis of the thiol ester substrates, or by measuring the efficiency of acylation of the active serine, using conventional techniques as described in the article in the names of Zhao et al., J. Bacteriol., 1997, 179, 4901-4908.

According to another advantageous embodiment of the invention, said identification is carried out by a method comprising at least the following steps:
a₂) preparing crystals from a mini-PBP2x as defined above,
b₂) determining the three-dimensional structure of said mini-PBP2x from the crystal obtained in a₂), and
c₂) identifying active substances capable of binding to the mini-PBP2x and/or of inhibiting the activity of said mini-PBP2x, which can be used as antibiotics.

In accordance with the invention:
the crystal is prepared from a free mini-PBP2x derived from a PBP2x from a β-lactam-sensitive or -resistant strain of S. pneumoniae, or else from mini-PBP2x/ligand complexes, by the suspended drop technique;
the three-dimensional structure of the mini-PBP2x is determined by conventional techniques known in themselves, such as nuclear magnetic resonance and X-ray diffraction;
the PBP2x inhibitors are identified by modelling of the structure of the free mini-PBP2x or of the mini-PBP2x/ligand complexes.

Advantageously:
the mini-PBP2x according to step a₂) is subjected to a prior treatment comprising a step of denaturation by temperature or by a chemical agent, in the presence or absence of ligands, followed by a step of renaturation under suitable conditions;
the mini-PBP2x according to step a₁) or a₂) consists of the concatenation of the fragments, as defined above, of a PBP2x from a β-lactam-resistant strain of S. pneumoniae; and
the detection carried out in step b₁) or the determination and identification carried out respectively in steps b₂) and c₂) are carried out by comparison with a mini-PBP2x consisting of the concatenation of the fragments, as defined above, of a PBP2x from a β-lactam-sensitive strain of S. pneumoniae.

A subject of the invention is also a kit for implementing the methods as defined above, characterized in that it includes at least one protein, one peptide, one antibody, one vector, one cell, one probe or one primer, as defined above.

The mini-PBP2x according to the invention, which can be produced in large amounts in a soluble functional form which is readily crystallizable, for all the PBP2xs, has the following advantages:
it is suitable for systematic screening of novel antibiotic molecules using functional assays (high throughput screening),
it is suitable for the structure-function study of the β-lactam-resistant variants of PBP2x and for the rational design of novel antibiotic molecules (molecular modelling or drug design).

Besides the arrangements above, the invention also comprises other arrangements, which will emerge from the following description, which refers to examples of use of the mini-PBP2x which is the subject of the present invention and also to Table I which summarizes the applicant's sequences and to the attached drawing, in which:

FIG. 1 illustrates the amino acid sequence of a mini-PBP2x (SEQ ID No. 1), derived from the PBP2x of the β-lactam-sensitive S. pneumoniae strain R6 (SWISSPROT P14677); the amino acids of PBP2x which have been deleted in the mini-PBP2x are replaced with a dash and those of the peptide fragments which have been inserted are represented in italics; the units specific to the class B PBPs are underlined.

TABLE I
Sequence listing

| Identification number | Sequence |
|---|---|
| SEQ ID No. 1 | Mini-PBP2x derived from PBP2x of |
| | the strain R6 |
| | (SWISSPROT P14677) |
| SEQ ID No. 2 | Primer 5′ ICNter |
| SEQ ID No. 3 | Primer 3′ ICCter |
| SEQ ID No. 4 | Oligonucleotide mini 1 |
| SEQ ID No. 5 | Oligonucleotide mini 2 |
| SEQ ID No. 6 | Oligonucleotide mini 3 |
| SEQ ID No. 7 | Oligonucleotide mini 4 |
| SEQ ID No. 8 | Oligonucleotide mini2x NdeI |
| SEQ ID No. 9 | Oligonucleotide mini2x XhoI |

It should be clearly understood, however, that these examples are given only by way of illustration of the subject of the invention, of which they in no way constitute a limitation.

### EXAMPLE 1

### Production of a Recombinant MINI-PBP2x

1) Construction of a Vector for Expression of a mini-PBP2x

a) Materials and Methods

A vector for expression of a mini-PBP2x was constructed from the plasmid pGEX-S-PBP2x*-fl (Mouz et al., J. Biol. Chem., 1999, 274, 19175-19180) containing the sequence encoding the PBP2x* of the β-lactam-sensitive S. pneumoniae strain R6 (fragment 49-750 of the PBP2x of the GENBANK amino acid sequence P14677, corresponding to the GENBANK nucleotide sequence X16367).

The deletions of the amino acids located between positions 49 to 73, 94 to 183, 200 to 217 and 230 to 256 and the insertion, in place of said deletions, respectively of the linking fragments Gly-Ser-Gly, Gly-Gly, Gly and Gly-Gly-Gly, was carried out by site-directed mutagenesis according to the method of Kunkel et al. (P.N.A.S., 1985, 82, 488-492), using the protocols as described in Mouz et al. (J. Biol. Chem., 1999, 274, 19175-19180).

More precisely, the phagemide called pGEX-S-PBP2x*-f1 is converted to single strand and then used as a matrix for the mutagenesis steps (deletions and insertions). The mutagenesis steps were carried out in two stages, the first with the oligonucleotides Mini 1 (SEQ ID No. 4) and Mini 3 (SEQ ID No. 6) and the second with the oligonucleotides Mini 2 (SEQ ID No. 5) and Mini 4 (SEQ ID No. 7):

Mini 1:
5′-CATAAATAGTCCCACGTTTGGCCCCGGATCCACGCGGAACCAG-3′,

this oligonucleotide makes it possible to delete the nucleotide sequence corresponding to the amino acids located between positions 49 and 73 of PBP2x and to insert in place of said deletion a linking fragment corresponding to a peptide Gly-Ser-Gly.

Mini 2:
5′-GTTTGGGTAACTACGATTGGGACCTCCAGAGGTTGCATCCTCAGC
AATCGG-3′,

this oligonucleotide makes it possible to delete the nucleotide sequence corresponding to the amino acids located between positions 94 and 183 of PBP2x and to insert in place of said deletion a linking fragment corresponding to a peptide Gly-Gly.

Mini 3:
5′-GTTCAAGGAACTCTCCATTCCACCGCCGATAAAACTAGAAGC
AAATTG-3′,

this oligonucleotide makes it possible to delete the nucleotide sequence corresponding to the amino acids located between positions 200 and 217 of PBP2x and to insert in place of said deletion a linking fragment corresponding to a peptide Gly.

Mini 4:
5′-TGTATAAACATCCTTACCGTCCCCACCTCCCCCTGCAAGAATA
CTGTTC-3′,

this oligonucleotide makes it possible to delete the nucleotide sequence corresponding to the amino acids located between positions 230 and 256 of PBP2x and to insert in place of said deletion a linking fragment corresponding to a peptide Gly-Gly-Gly.

The plasmid thus obtained, named pGEX-S-mini-PBP2x-f1, was used as a matrix to PCR amplify the DNA fragment corresponding to the mini-PBP2x and to introduce, at its ends, the NdeI and XhoI restriction sites, for cloning into the commercial vector pET30b (NOVAGEN). The oligonucleotides used for the PCR amplification are as follows:

oligo Mini2x NdeI:

(SEQ ID No. 8)

5′-CCGCATATGGCCAAACGTGGGACTATTTAT-3′
oligo Mini2x XhoI:

(SEQ ID No. 9)

5′-GGCTCGAGTTAGTCTCCTAAAGTTAATGTAAT-3′

The nucleotide sequence of the mini-PBP2x, in the expression vector thus obtained, named pET30b-mini-PBP2x, was confirmed by automatic sequencing.

b) Results

The peptide sequence deduced from the nucleotide sequence obtained by automatic sequencing has the sequence SEQ ID No. 1 (FIG. 1), corresponding to that expected for a mini-PBP2x.

The mini-PBP2x consists of the succession of fragments corresponding respectively to the amino acids located between positions 74 to 93, 184 to 199, 218 to 229 and 257-750 of PBP2x (SWISSPROT accession number P14677), each fragment being preceded, respectively, by the linking fragment GSG, GG, G and GGG.

2) Production and Purification of mini-PBP2x

a) Materials and Methods

The mini-PBP2x is produced in E. coli, using the expression vector described in Example 1.1, and it is then purified by chromatography, successively on a Q-Sepharose, Resource Q and Superdex 200 column. The product obtained is analysed by polyacrylamide gel electrophoresis (SDS-PAGE) and by mass spectrometry (Electrospray ionization-mass spectrometry, ESI-MS).

More precisely, 2 litres of culture of the E. coli strain BL21 (DE 3), transformed with the plasmid pET30b-mini-PBP2x, are induced at an optical density of 1 (600 nm), for 15 h at 16° C. After centrifugation, the bacterial pellet is washed with 500 ml of buffer A (20 mM Tris HCl, pH 8.0, 20 mM NaCl, 1 mM EDTA) containing protease inhibitors, and is then resuspended in 50 ml of the same buffer.

After sonication (6 min on an ice/ethanol bed), the 50 ml of bacterial lysate are centrifuged at 40 000 g, at +4° C., for 20 min, and the supernatant obtained is loaded onto a 20 ml ion exchange column (Q-Sepharose, AMERSHAM-PHARMACIA), pre-equilibrated with buffer A. A linear sodium gradient is produced with buffer B (20 mM Tris HCl, pH 8.0, 300 mM NaCl, 1 mM EDTA). The mini-PBP2x protein is eluted from 38% buffer B. The eluted fractions are analysed by Western blotting using rabbit polyclonal antibodies directed against PBP2x*, and are then pooled together. The pool of fractions containing the mini-PBP2x is diluted 10-fold in buffer A without NaCl, and is then loaded onto a 6 ml Resource Q column (AMERSHAM-PHARMACIA) pre-equilibrated with buffer A. A linear sodium gradient is produced with the buffer. The mini-PBP2x protein is eluted from 55% of buffer B. The fractions corresponding to the elution peak are analysed by SDS-15% PAGE. The purest fractions are pooled together and the pool obtained is concentrated to a volume of 2 to 4 ml, and then loaded onto a gel chromatography column (Superdex 200, 16/60, AMERSHAM-PHARMACIA), pre-equilibrated in a 10 mM Hepes buffer, pH 7.5, 100 mM NaCl, 1 mM EDTA. The mini-PBP2x is eluted in a symmetrical peak at an elution volume corresponding to an apparent mass of 60 kDa.

b) Results

The preparation of mini-PBP2x protein thus obtained has a degree of purity greater than 95%. The final yield of purified mini-PBP2x protein is 10 to 15 mg/litre of culture, depending on the preparations.

The purification by gel chromatography shows the presence of a symmetrical elution peak which reflects the homogeneity of the protein in terms of oligomerization. The elution volume corresponds to a monomeric protein.

The mini-PBP2x protein exhibits excellent solubility since it can be concentrated to 15 mg/ml without precipitating.

The analysis by mass spectrometry shows the presence of a homogeneous protein which has a molecular mass of 59 465 Da corresponding to that calculated from the amino acid sequence.

### EXAMPLE 2

### Analysis of the Physicochemical and Enzymatic Properties of the MINI-PBP2x

1) Materials and Methods

The physicochemical and enzymatic properties of the purified mini-PBP2x obtained in Example 1 were measured in a similar way to those of the variants of PBP2x, as described in Mouz et al. (P.N.A.S., 1998, 95, 13403-13406).

More precisely:

a) Molecular Weight

The molecular weight was determined by ESI-MS (electrospray ionization-mass spectrometry).

b) Isoelectric Point

The theoretical isoelectric point was determined from the amino acid composition of the mini-PBP2x.

c) Extinction Coefficient

The molar extinction coefficient was determined experimentally by measuring inhibition of the mini-PBP2x by cefotaxime, or theoretically from the amino acid sequence.

d) Acylation Kinetics

The efficiency of acylation of the mini-PBP2x by β-lactams, which is defined by the value k₂/K where K=k₁/k according to equation 1 (Eq1), was determined, in the presence of cefotaxime (3rd generation antibiotic); the interaction of the PBPs (enzyme E) with the β-lactams (I) is represented by the equation Eq1, in which EI, EI* and P represent, respectively, the Michaelis-Menten complex, the acyl-enzyme covalent complex and the product (degraded β-lactam):

$$E + I {{{k}_{1}}^{}}_{{k}_{1}} EI {->}^{{k}_{2}} {EI}^{*} {->}^{{k}_{3}} E + P . \qquad ( Eq 1 )$$

e) Hydrolysis Kinetics

The transpeptidase activity (hydrolytic activity) of the mini-PBP2x, defined by the value kcat/Km, was determined in the presence of two PBP2x substrate analogues [N-benzoyl-D-alanylmercaptoacetic thiol ester (S2d) and carboxymethylbenzoylamino-thioacetate thiol ester (S2a)], synthesized according to the protocol described in Adam et al. (Biochem. J., 1990, 270, 525-529).

2) Results

The physicochemical characteristics of the mini-PBP2x are given in Table II below.

TABLE II
Physicochemical parameters of the mini-PBP2x

| Parameter | Mini-PBP2x |
|---|---|
| Molecular weight (daltons) | 59 465 ± 5 |
| Theoretical isoelectric point | 4.5 |
| Extinction coefficient at 280 nm | 52 830 ± 50 |
| (mol⁻¹ cm⁻¹ L⁻¹) | |

The kinetic parameters for the mini-PBP2x are given in Table III below, by comparison with those of PBP2x* (Jamin et al., Biochem. J., 1993, 292, 735-741; Mouz et al., P.N.A.S., 1998, 95, 13403-13406).

TABLE III
Compared kinetic parameters of mini-PBP2x and of PBP2x*

| Parameters¹ | Mini-PBP2x | PBP2x*² | PBP2x*³ |
|---|---|---|---|
| k₂/K | 146 100 ± 9 000 | 162 000 ± 400 | 209 000 ± 1 800 |
| (cefotaxime) | | | |
| kcat/Km | 76 ± 3 | 610 ± 150 | 139 ± 8 |
| (S2a) | | | |
| kcat/Km | 2 890 ± 400 | 5 000 ± 1 400 | 2 500 ± 200 |
| (S2d) | | | |

¹Expressed as M⁻¹ · s⁻¹,
²Jamin et al., Biochem. J., 1993, 292, 735-741,
³Mouz et al., P.N.A.S., 1998, 95, 13403-13406

Analysis of the kinetic parameters of the variants of PBP2x shows that, by comparison to PBP2x*, from which only the cytoplasmic and transmembrane domains of PBP2x are deleted (residues 1 to 48), the mini-PBP2x, which has additional deletions in the n-PB domain (non penicillin-binding domain), has enzymatic properties (binding to β-lactams and transpeptidase activity) equivalent to those of PBP2x*.

### EXAMPLE 3

### Crystallization of a MINI-PBP-2x

1) Materials and Methods

The mini-PBP2x is purified as described in Example 1. The conditions for crystallization of the mini-PBP-2x, obtained by the suspended drop method, are as follows: mini-PBP2x (12 mg/ml), 100 mM sodium Hepes, pH 7.5, 2% V/V PEG 400, 2M ammonium sulphate, at a temperature of 8° C.

2) Results

The crystals appear after a few weeks; they exhibit a hexagonal bipyramidal form with a length of the order of 150 μm.

### EXAMPLE 4

### Analysis of the Diffraction Properties of a MINI-PBP2x

1) Materials and Methods

The diffraction data was recorded at the ESRF (European Synchrotron Radiation Facility, Grenoble, France). More precisely, the crystals were immersed in the crystallization buffer as described in Example 3, supplemented with ethylene glycol (10% V/v) before being rapidly cooled in liquid nitrogen. The diffraction data was recorded on the ID14-EH2 beam line and processed using the MOSFLM and SCALA programs of the CCP4 suite (Acta Crystallograph. Sect. D, 1994, 54, 905-921).

2) Results

The crystals which diffract up to 2.5 Å exhibit a hexagonal elemental cell (a=b=136.4 Å, c=142.8 Å) and belong to the space group P6₁/P6₅, with two molecules in the asymmetric unit. The results of a complete diffraction set collected at the ESRF are given in Table IV below, by comparison with the data previously obtained with soluble PBP2x* (Dessen et al., J. Biol. Chem., 2001, 48, 45106-45112; Gordon et al., J. Mol. Biol., 2000, 299, 477-485).

TABLE IV
Compared crystallography data for mini-PBP2x and for PBP2x*

| Crystallography | | PBP2x* | PBP2x* |
|---|---|---|---|
| data | Mini-PBP2x | (Dessen et al.) | (Gordon et al.) |
| Dimensions (Å) | a = b = 136.4, | a = b = 146.56, | a = b = 129.9, |
| | c = 142.8 | c = 132.61 | c = 139.86 |
| Space group | P6₁/P6₅ | P3₂ | P4₁2₁2 |
| Minimum resolution | 2.5 | 3.2 | 2.4 |
| (Å) | | | |
| Number of unique | 51 815 | 52 413 | 42 234 |
| reflexions | | | |
| Rsym (%) | 10 (29.6)* | 11.1 (36.7)* | 6.3 (36.8)* |
| I/σI | 2.3 | 10.2 (3.1)* | |
| Redundancy | 4.0 | 3.2 | 7 |
| Completion (%) | 99.9 | 96.1 (97.8)* | 99.7 (93.5)* |

*Corresponding value in the final layer of diffraction.

Table IV shows that the diffraction data from mini-PBP2x amount to 51 815 unique reflexions with an Rsym of 10% (30% in the final layer of diffraction: 2.64 to 2.5 Å), with a degree of completion of 99.9%.

A finer analysis of the data obtained with the mini-PBP2x, as a function of the resolution, is given in Table V below; the most significant data are indicated in bold.

TABLE V
Statistical analysis of the diffraction data
for mini-PBP2x as a function of resolution

| Dmin¹ | Rfac² | Rfull³ | Rcum⁴ | Av-I⁵ | σ⁶ | I/σ | Nmea⁷ | Nref⁸ |
|---|---|---|---|---|---|---|---|---|
| 7.91 | 0.069 | 0.070 | 0.069 | 39 616 | 5 195.0 | 7.6 | 4 938 | 1 556 |
| 5.59 | 0.085 | 0.080 | 0.077 | 18 912 | 2 986.4 | 6.3 | 10 812 | 3 027 |
| 4.56 | 0.092 | 0.075 | 0.085 | 26 060 | 4 275.6 | 6.1 | 14 633 | 3 889 |
| 3.95 | 0.087 | 0.056 | 0.086 | 26 842 | 4 120.7 | 6.5 | 18 118 | 4 617 |
| 3.54 | 0.084 | 0.063 | 0.085 | 16 584 | 2 210.7 | 7.5 | 20 977 | 5 210 |
| 3.23 | 0.094 | 0.067 | 0.086 | 9 999 | 1 434.4 | 7.0 | 23 488 | 5 776 |
| 2.99 | 0.125 | 0.105 | 0.089 | 5 454 | 996.0 | 5.5 | 25 470 | 6 242 |
| 2.80 | 0.165 | 0.136 | 0.092 | 3 227 | 771.4 | 4.2 | 27 395 | 6 710 |
| 2.64 | 0.223 | 0.179 | 0.096 | 2 006 | 648.9 | 3.1 | 29 137 | 7 145 |
| 2.5 | 0.296 | 0.220 | 0.100 | 1 378 | 594.1 | 2.3 | 30 891 | 7 580 |

¹minimum resolution (Å),
²Confidence factor in the range,
³Confidence factor for full reflexions on a film,
⁴Cumulative confidence factor,
⁵Mean of intensities <I>,
⁶Standard deviation of intensities,
⁷Number of reflexions measured,
⁸Number of unique reflexions in the resolution range.

The data given in Tables IV and V above show that the mini-PBP2x makes it possible to obtain crystals with a good diffraction power (2.5 Å).

### EXAMPLE 5

### Screening of Antibiotic Molecules using a MINI-PBP2x

The screening of inhibitors of a mini-PBP2x, which can be used as antibiotics, is carried out using binding assays or using assays for inhibition of the enzymatic activity (hydrolytic activity) of the mini-PBP2x, in the presence of the test molecule.

1) Mini-PBP2x-Binding Assay

It has been shown that the intrinsic fluorescence of PBP2x decreases during the binding, at the active site, of inhibitors such as β-lactams (Jamin et al., Biochem. J. 292, 735-741). Consequently, mini-PBP2x inhibitors which can be used as antibiotics are screened in a similar way.

More precisely, the mini-PBP2x, prepared as described in Example 1, is distributed into the wells of a plate (2 to 500 μl of mini-PBP2x in 10 mM phosphate buffer, pH 7), and the test molecules are then added and the plates are incubated at a temperature of between 5° C. and 40° C. for a period of between 30 s and 1 h. The variation in fluorescence of the mini-PBP2x, in the presence or absence of test molecule, is measured in a wavelength window of between 305 and 360 nm, after excitation at a wavelength of 280 nm; said measurement is made either continuously or after various incubation times of between 30 s and 1 h. The molecules capable of binding at the active site of the mini-PBP2x, corresponding to those for which a decrease in the intrinsic fluorescence of the mini-PBP2x is observed, are selected.

2) Assay for Inhibition of the Enzymatic Activity of the mini-PBP2x

The hydrolysis of pseudosubstrates of thioester type [N-benzoyl-D-alanylmercaptoacetic thiol ester (S2d) and carboxymethylbenzoylaminothioacetate thiol ester (S2a)] is measured with the mini-PBP2x, alone or in the presence of test molecule, according to the protocols as described in Zhao et al., mentioned above. The molecules capable of inhibiting the hydrolytic activity of the mini-PBP2x are selected.

1-22. (canceled)

23. A recombinant protein obtained from Streptococcus pneumoniae PBP2x protein, which recombinant protein comprises concatenated fragments corresponding, respectively, to amino acids located between positions 74 to 90, 186 to 199, 218 to 228 and 257 to 750, with reference to the sequence of the PBP2x protein of the strain R6 (SWISSPROT p 14677 or GENBANK 18266817), each one of said fragments being preceded by a peptide fragment of 1 to 7 amino acids.

24. The recombinant protein of claim 23, wherein the peptide fragment comprises amino acids of said Streptococcus pneumoniae PBP2x protein located between positions −1 to −7, relative to the residues at positions 74, 186, 218 and 257, or between positions +1 to +7, relative to the residues at positions 90, 199 and 228, as defined in claim 23, or both.

25. The recombinant protein of claim 23, wherein the peptide fragment comprises amino acids comprising alanine (A), serine (S), glycine (G) or threonine (T) or a combination thereof.

26. The recombinant protein of claim 23, which is obtained from a β-lactam-resistant strain of S. pneumoniae.

27. The recombinant protein of claim 23, which has the sequence of SEQ ID No. 1.

28. The recombinant protein of claim 23, which comprises a substitution of at least one methionine residue with a selenomethionine residue.

29. The recombinant protein of claim 23, which is associated with a ligand.

30. The recombinant protein of claim 23, which is in the form of a crystal.

31. The recombinant protein of claim 23, wherein said Streptococcus pneumoniae PB2x protein has a sequence identity which is at least 30% identical with the sequence of the strain R6 (SWISSPROT p 14677).

32. The recombinant protein of claim 31, wherein said Streptococcus pneumoniae PB2x protein has a sequence identity which is at least 50% identical with the sequence of the strain R6 (SWISSPROT p 14677).

33. A peptide which comprises a fragment of at least 7 amino acids of the mini-PBP2x protein of claim 23, which peptide includes at least one residue comprising those located at positions 74, 90, 186, 199, 218, 228 and 257 as defined in claim 24.

34. Antibodies which are directed against the peptide of claim 33.

35. An isolated nucleic acid molecule, which is selected from the group consisting of the sequences encoding a mini-PBP2x of claim 23, and sequences complementary to the preceding sequences, which are sense or antisense.

36. A pair of primers, having the sequence of SEQ ID Nos. 2-3.

37. Primers comprising a sequence of approximately 10 to 30 nucleotides corresponding to those located at the junction of the peptide fragments of 1 to 7 amino acids and the fragments of PBP2x of claim 23.

38. The primers of claim 37, having a sequence selected from the group consisting of the sequences of SEQ ID Nos. 4 to 9.

39. A recombinant vector, which comprises an insert selected from the group consisting of the nucleic acid molecules encoding a mini-PBP2x of claim 35.

40. The recombinant vector of claim 39, which is a prokaryotic vector.

41. Cells transformed with the recombinant vector of claim 39.

42. Cells transformed with the recombinant vector of claim 40.

43. The cells of claim 42, which are prokaryotic cells.

44. A method for screening antibiotics, which comprises at least the following steps:
a₁) bringing a mini-PBP2x of claim 23 into contact with a test compound,
b₁) detecting binding of said test compound with the mini-PBP2x or inhibiting activity of the mini-PBP2x resulting from the binding or both, and
c₁) identifying active compounds capable of binding to the mini-PBP2x or of inhibiting the activity of the mini-PBP2x, or both, for use of the active compounds as antibiotics.

45. The method of claim 44, wherein said inhibited activity of the mini-PBP2x in steps b₁) and c₁) is inhibited hydrolytic activity.

46. A method for identifying antibiotics, which comprises at least the following steps:
a₂) preparing crystals from a mini-PBP2x of claim 23,
b₂) determining a three-dimensional structure of the mini-PBP2x from the crystals obtained in a₂), and
c₂) identifying active compounds capable of binding to the mini-PBP2x or of inhibiting the activity of the mini-PBP2x, or both, for use as antibiotics.

47. The method of claim 46, wherein said inhibited activity of the mini-pB2x in step c₂) is inhibited hydrolytic activity.

48. A screening kit, which comprises at least one protein, one peptide, one antibody, one vector, one cell, one probe or one primer of claim 23.
